(19) United States
(12) Patent Application Publication
(10) Pub. No.: US 2005/0279469 A1
(43) Pub. Date: Dec. 22, 2005

(54) Retention aid additives for paper making

(76) Inventors: Bradley A. Saville, Toronto (CA); Robert Pelton, Dundas (CA)

Correspondence Address: MANELLI DENISON & SELTER, 2000 M STREET NW SUITE 700, WASHINGTON, DC, 20036-3307, US

(21) Appl. No.: 10/871,031

(22) Filed: Jun. 21, 2004

(51) Int. Cl. D21C 3/20; C12N 9/42; C12N 11/04

(52) U.S. Cl. 162/72; 435/182; 435/209

(57) ABSTRACT

A selective immobilized enzyme bioadditive retention aid comprising an enzyme chemically bound to a particulate inorganic support, said enzyme having a binding site selectively linkable to an organic polymeric fibrous material component of a pulp. The invention provides an improved process of paper manufacture and the novel paper making additives of use therein.

### FIELD OF THE INVENTION

This invention relates to paper making processes and additives of use therein.

### BACKGROUND OF THE INVENTION

Modern papers are a sophisticated blend of fibers, fillers and polymers wherein during manufacture synthetic retention aids are required to enhance the deposition of fillers and fines onto the fibers before sheet formation. Without retention aids, most of the fillers and some of the fines pass through the sheet and remain in the white water. Poor retention lowers product quality and paper-machine efficiency, and wastes raw material. Loss of fines is especially significant in thermomechanical and chemi-thermomechanical pulping processes, which produce a significant quantity of fines in suspension.

Typical retention aids include high molecular weight organic polymers, with either positive, negative or net neutral charges. Cationic polymers are frequently employed to bind with cellulosic fibres, which naturally possess a negative charge. Unfortunately, conventional “effective” retention aids cause everything to stick to everything else. Fillers form large aggregates instead of uniformly depositing onto fibers and unwanted fiber-fiber flocculation gives poor sheet formation. A fundamental problem is that retention aid adsorption is not selective in that fines, fillers and fibers compete for the retention additives.

Pulp and Paper Canada, 81, 54, 1980—R. H. Pelton, L. H. Allen, and H. M. Nugent and TAPPI J, 68, 91, 1985—R. H. Pelton describe the use of high molecular weight organic polymers as retention aids for pulp systems, and compare the effects of charge on retention aid effectiveness.

“Pulp and Paper: Chemistry and Chemical Technology, Vol 2”, Chap 8, T. N. Kershaw, ed., John Wiley and Sons, New York, 1981—J. P. Casey describes the impact of the loss of cellulosic fines on paper properties, the justification for the need for retention aids for effective paper formation and the economic impact of fine fiber loss.

“Handbook for Pulp and Paper Technologies”, Chap 15, CPPA/TAPPI joint textbook committee, 1982—G. A. Smook describes, in a general way, the role of retention aids in papermaking in that they facilitate the aggregation of fibers and fine materials of cellulosic fines or fillers, which are often less than about 5 microns.

J. Wood Chem. Technol., 9, 407, 1989—S. Roy, M. Desrochers, and L. Jurasek describes the use of proteins as potential retention aids, including lysozyme, trypsinogen, myoglobin, carbonic anhydrase and protease. Proteins were chosen based on their charge characteristics, without any consideration of their ability to bind chemically to the materials in a papermaking suspension, i.e., the expected mechanism of action for these proteins was essentially equivalent to that observed with high molecular weight polymers currently used in industry. However, the results suggested that, in addition to electrostatic interactions, some other form of binding was probably also occurring. The isoelectric point of the proteins had a significant effect on binding, suggesting that most of the binding was based on electrostatic interactions.

U.S. Pat. No. 5,998,183, issued Dec. 7, 1999—G. N. Le Fevre and B. A. Saville, describes the immobilization of enzymes, and means to ensure that enzyme activity is retained and maximized. The teachings of the processes for making immobilized enzymes described in U.S. Pat. No. 5,998,183 is incorporated herein by reference.

### SUMMARY OF THE INVENTION

It is an object of the present invention to provide an improved process of paper manufacture and novel paper making additives of use therein.

Generally, the invention provides processes of preparing selective paper making additives by immobilizing enzymes, for example, cellulase/cellulose binding domain (CBD) onto a clay/kaolinite support, and/or by co-immobilizing cellulase/(CBD) and α-amylase onto a clay/kaolinite support. The cellulase/CBD moiety selectively binds to cellulose in a papermaking suspension, while amylase will bind to starch. The product obtained is therefore highly selective, possessing specific recognition sites for starch and cellulose. Furthermore, the immobilization support (clay/kaolinite) is a commonly used filler within paper.

Accordingly, in one aspect, the invention provides a selective immobilized enzyme bioadditive retention aid comprising an enzyme chemically bound to a particulate inorganic support, said enzyme having a protein recognition site selectively linkable to an organic polymeric fibrous material component of a pulp.

Preferably, the enzyme having the protein recognition site is selected from the group consisting of cellulase, a cellulose binding domain, amylase, mannanase and xylanase.

The particulate support is preferably a paper-making acceptable filler or coating material, such as a paper-making acceptable clay and most preferably, kaolinite.

The fibrous material is preferably selected from cellulose, hemi-cellulose, xylan, mannan and lignan.

In a preferred aspect the retention aid has a dual functionality in containing the protein recognition sites of cellulase and amylase.

In a further aspect, the invention provides a method of preparing a paper sheet from a pulp comprising an organic polymeric fibrous material, said method comprising treating said pulp with a retention aid to assist in enhancing the deposition of said fines onto said fibers to produce a resultant pulp and forming said paper sheet from said pulp; the improvement wherein said retention aid comprises an enzyme chemically bound to a particulate inorganic support, said enzyme having a protein recognition site selectively linkable to an organic polymeric fibrous material component of said pulp.

The pulp may be a slurry comprising significant volumes of water, or in an alternative embodiment, be in the form of a damp sheet of paper-forming components, wherein the process comprises coating one or both sides of the sheet with a retention aid according to the invention.

In preferred embodiments for coating a sheet, starch fines in admixture with an amylase-containing retention aid according to the invention with, optionally, also a immobilized cellulase is used.

In a yet further aspect, the invention provides a paper sheet comprising fibers, fines, a filler and a retention aid wherein said retention aid is a an enzyme chemically bound to a particulate inorganic support, said enzyme having a protein recognition site selectively linkable to an organic polymeric fibrous material component of a pulp.

### BRIEF DESCRIPTION OF THE DRAWINGS

In order that the invention may be better understood, preferred embodiments will now be described by way of example only with reference to the accompanying drawings, wherein

FIG. 1 is a graph showing immobilized enzyme activity for different loadings of a clay;

FIG. 2 represents scanning election microscope images of clay on cotton fibers;

FIG. 3 is a graph showing the production of xylo-oligosaccharides with time using xylanase immobilized on kaolinite; and

FIG. 4 is a diagrammatic sketch of a dynamic drainage jar apparatus.

### DETAILED DESCRIPTION OF PREFERRED EMBODIMENTS

Use of the process of the invention for starch coatings is described herein.

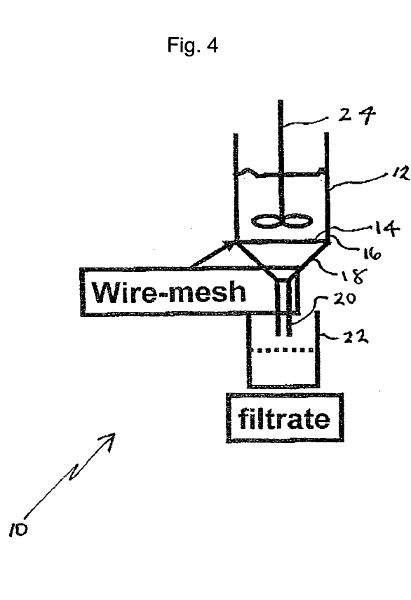

FIG. 4 shows generally as 10, a dynamic drainage jar apparatus used to compare absorption/retention values in several experiments using particulate clay supports.

Apparatus 10 is a right-vertical cylindrical glass vessel 12, which holds the pulp slurry under test, fitted with a wire mesh 14 at a lower part 16. Lower part 16 has a conical bottom 18 having a drainage spout 20, located within a recepticle 22. Vessel 12 has a paddle-stirrer 24.

Use of the DDJ apparatus is exemplified under Example 3, hereinafter.

Starch coatings (“sizes”) are typically utilized to fill in gaps or voids on the surface of the sheet. These coatings may be administered on a size press, or by tub sizing. In the former operation, the rollers (or nip) are flooded with starch solution, which is then delivered and attached to the paper by absorption as it passes between the rollers. The process must be carefully controlled to avoid non-uniform distribution of the sizing agent onto the paper stock. In particular, the paper machine may need to run at relatively low or moderate speeds to ensure proper attachment of the solids in the sizing solution. In a tub sizing operation, the sheet passes through a shallow tub or bath that contains the starch solution; excess solution is subsequently removed by passing the sheet through a set of rollers.

In either type of process, the objective is to achieve a uniform coating of the sizing agent onto the paper. Selective or controlled deposition of starch onto the underlying cellulosic component of the paper will improve sheet formation and coating uniformity. The additives described herein, which promote selective binding between paper constituents and starch therefore improves deposition of starch coatings onto paper.

Enzymes were immobilized onto clay/kaolinite, through a cross-linking process, adapting an existing immobilization technology, in accordance with aforesaid U.S. Pat. No. 5,998,183, to α-amylase, cellulase, and the cellulose-binding domain (CBD) of cellulase. The effectiveness of the intelligent paper additive was established through adsorption studies, followed by examination of fibers using scanning electron microscopy. Measurements of the activity of the soluble and immobilized enzyme provided additional evidence regarding the effectiveness of the immobilization procedure.

### EXAMPLES

### Example 1

### Experiments with Cellulase Immobilized on Kaolinite

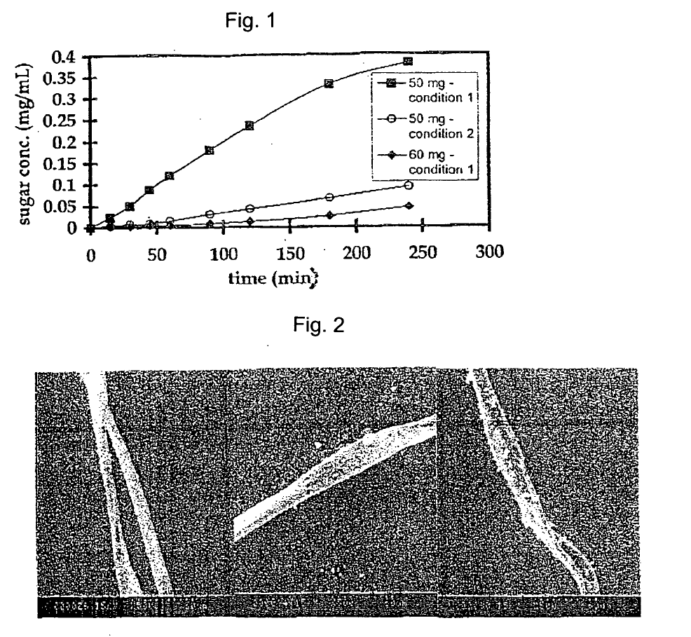

For a variety of different immobilization conditions, measurements of the activity of soluble cellulase before and after immobilization were conducted, based on the production of reducing sugars. The activity of the immobilized enzyme was affected by the duration of immobilization, and the relative concentration of cellulase to clay. For the support modification step, which takes place over 1.5 to 10 hours, with glutaraldehyde, the concentration typically ranged between 1 and 4% (v/v). For the immobilization step, the modified support was incubated in enzyme solution, comprised of raw enzyme solution diluted either 5, 10, or 20 fold, to establish the effect of protein concentration on the immobilization process. The activity of the immobilized enzyme for some different loadings of clay and immobilization conditions is shown in FIG. 1. These data indicate that cellulase is attached to clay, and that this configuration recognizes the cellulose in the solution, establishing that cellulose and clay can be attached in this manner.

Adsorption studies with the immobilized cellulase (bioadditive) were conducted by incubating the bioadditive in a solution containing cotton (cellulose) fibers. As a control, fibers were also incubated in a suspension of clay alone. FIG. 2 compares the SEM images of native cotton fibers, cotton fibers exposed to clay only, and cotton fibers incubated in cellulase immobilized onto clay, wherein

(a) is untreated cotton fiber;

(b) is cotton fiber incubated in clay suspension;

is cotton fiber incubated in clay-cellulase complex

As shown in FIG. 2, much more clay is attached to the cotton when the clay is coupled to cellulase than if the cotton is incubated in clay alone. The selective interaction between cellulase and cellulose (cotton) thus facilitates greater deposition of clay.

### Example 2

### Experiments with Xylanase Immobilized on Kaolinite

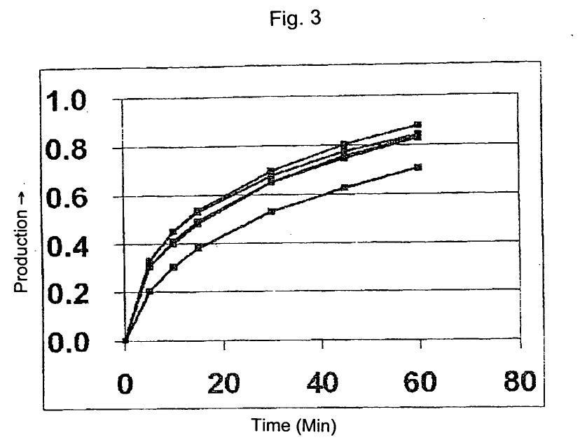

Kaolinite (clay) was suspended in 200 mL of glutaraldehyde/phosphate buffer (pH 7.5) solution for 2 to 8 hours, then recovered by vacuum filtration. The recovered kaolinite was washed with 600 mL of distilled water, and dried. The dried modified support was incubated for 4 to 24 h in enzyme solution, prepared by diluting raw xylanase, either 5, 10 or 20 fold, with phosphate buffer (pH 7.5). The immobilized enzyme was recovered by vacuum filtration and dried. Activity tests using birchwood xylan confirmed that the xylanase attached to the kaolinite was active (FIG. 3).

### Example 3

### Retention Studies

Adsorption/retention studies were conducted using a dynamic drainage jar (DDJ) apparatus (FIG. 4) to compare retention of unmodified clay, cellulase immobilized on clay (from Example 1), and xylanase immobilized on clay (from Example 2):
1. 8.0 g of unbeaten bleached kraft softwood pulp was put in deionized water for 24 hours, and diluted to 2 L with deionized water.
2. The pulp slurry was disintegrated at 15000 revolutions, and then diluted to 4 L with deionized water.
3. 250 mL of pulp slurry was mixed with 200 mL water, put into the DDJ apparatus and mixed at 800 rpm.
4. 0.25 g of filler sample (either unmodified clay or clay modified with enzyme) was suspended in 10 mL of water, then added into the DDJ apparatus and mixed for two minutes.
5. The containers originally containing the pulp solution and the filler solution were rinsed using 40 mL of water, which was then transferred into the DDJ apparatus.
Thus, at the conclusion of steps (1)-(5), the DDJ apparatus contained 500 mL of water, 0.5 g of pulp, and 0.25 g of filler.
6. After the two minutes had elapsed, the DDJ mixer was turned off.
7. 20-30 mL of slurry was drained off the bottom of the DDJ apparatus and discarded. The next 100 mL was collected for filtration analysis, and the rest was drained completely, to collect a fiber cake formed on the screen of the DDJ apparatus.
8. The 100 mL of filtrate collected in step (7) was vacuum filtered using 0.22 micron filter paper. The filter paper was collected after filtration, and dried. The mass of solid collected was determined by comparing the dry mass of the filter paper before and after the filtration step; this information was subsequently used to calculate the first pass retention of pulp and filler on the mesh.
9. Ash/pyrolysis tests were performed on the filter+fibre cake collected during step (7), to determine the relative amount of (non-pyrolyzable) filler versus (pyrolyzable) fibre in the sample.

Table 1 shows that the first-pass retention of filler (clay) was improved by coupling with cellulase or xylanase.

TABLE 1

| | First pass retention, |
|---|---|
| Filler | % |
| Clay | 2.3 |
| Cellulase- | 5.2 |
| modified clay | |
| Xylanase- | 13.3 |
| modified clay | |

Table 2 also shows that modification of clay with enzymes, particularly xylanase, substantially improves the ash (filler) content of pulp samples retained on the DDJ mesh.

TABLE 2

| Filler | Filler on Fibre (g/g) |
|---|---|
| Clay | 0.0128 |
| Cellulase- | 0.0096 |
| modified clay | |
| Xylanase- | 0.0252 |
| modified clay | |

Although this disclosure has described and illustrated certain preferred embodiments of the invention, it is to be understood that the invention is not restricted to those particular embodiments. Rather, the invention includes all embodiments which are functional or mechanical equivalents of the specific embodiments and features that have been described and illustrated.

1. A selective immobilized enzyme bioadditive retention aid comprising an enzyme chemically bound to a particulate inorganic support, said enzyme having a binding site selectively linkable to an organic polymeric fibrous material component of a pulp.

2. A retention aid as defined in claim 1 wherein said enzyme is selected from the group consisting of cellulase, a cellulose binding domain, amylase, mannanase and xylanase.

3. A retention aid as defined in claim 1 comprising at least two of said enzymes.

4. A retention aid as defined in claim 3 comprising cellulase and amylase.

5. A retention aid as defined in claim 3 comprising xylanase and amylase.

6. A retention aid as defined in claim 3 comprising mannanase and amylase.

7. A retention aid as defined in claim 3 comprising cellulose binding domain and amylase.

8. A retention aid as defined in claim 1 wherein said support is selected from a paper making acceptable clay.

9. A retention aid as defined in claim 8 wherein said clay is kaolinite.

10. A retention aid as defined in claim 1 wherein said support is titanium dioxide.

11. A retention aid as defined in claim 1 wherein said fibrous material is selected from cellulose, hemi-cellulose, xylan, mannan and lignan.

12. A method of preparing a paper sheet from a pulp comprising an organic polymeric fibrous material, said method comprising treating said pulp with a retention aid to assist in enhancing the deposition of said fines onto said fibers to produce a resultant pulp and forming said paper sheet from said pulp; the improvement wherein said retention aid comprises an enzyme chemically bound to a particulate inorganic support, said enzyme having a binding site selectively linkable to an organic polymeric fibrous material component of said pulp.

13. A method as defined in claim 12 wherein said enzyme is selected from the group consisting of cellulase, a cellulose binding domain, amylase, mannanase and xylanase.

14. A method as defined in claim 12 wherein said support is selected from a paper making acceptable clay or titanium dioxide.

15. A method as defined in claim 14 wherein said clay is kaolinite.

16. A method as defined in claim 12 wherein said fibrous material is selected from cellulase, hemi-cellulose, xylan, mannan and lignan.

17. A method as defined in claim 12 wherein said pulp is a slurry.

18. A method as defined in claim 12 wherein said pulp is in the form of a sheet having a first side and a second side, and further comprising treating said sides with said retention aid to provide a resultant coated paper sheet.

19. A method as defined in claim 12 wherein said pulp further comprises a filler.

20. A method as defined in claim 19 wherein said filler is selected from the group consisting of a clay, kaolinite, titanium dioxide and calcium carbonate.

21. A method as defined in claim 12 wherein said retention aid comprises at least two of said enzymes.

22. A method as defined in claim 21 where said enzymes are cellulase and amylase.

23. A method as defined in claim 21 where said enzymes are xylanase and amylase.

24. A method as defined in claim 21 where said enzymes are mannanase and amylase.

25. A method as defined in claim 18 wherein said sheet has a cellulose component, said method further comprising treating said sheet with a slurry comprising starch slurry in the presence of said selective retention aid to effect selective binding between said starch slurry and said cellulose component and enhanced deposition of said starch slurry on said resultant coated paper sheet.

26. A paper sheet comprising fibers, fines, a filler and a retention aid wherein said retention aid is an enzyme chemically bound to a particulate inorganic support, said enzyme having a protein recognition site selectively linkable to an organic polymeric fibrous material component of a pulp.

27. A paper sheet as defined in claim 26 wherein said enzyme is selected from cellulase, a cellulose binding domain, amylase, and xylanase.

28. A coated paper sheet comprising a paper sheet as defined in claim 26 comprising fibers, fines, a filler and a retention aid wherein said retention aid is an enzyme chemically bound to a particulate inorganic support, said enzyme having a binding protein recognition site selectively linkable to an organic polymeric fibrous material component of a pulp.
